(19) United States
(12) Patent Application Publication
(10) Pub. No.: US 2010/0144896 A1
(43) Pub. Date: Jun. 10, 2010

(54) THIXOTROPIC PHARMACEUTICAL COMPOSITIONS

(76) Inventors: Mikyoung Hahn, Seoul (KR); Se-Hyun Song, Gyeonggi-do (KR); Hee-Jeong Lim, Gyeonggi-do (KR); Jin-Woo Choi, Seoul (KR); Hyeon Kuk, Gyeonggi-do (KR)

Correspondence Address: DICKSTEIN SHAPIRO LLP, 1825 EYE STREET NW, Washington, DC, 20006-5403, US

(21) Appl. No.: 12/527,120

(22) PCT Filed: Feb. 4, 2008
(86) PCT No.: PCT/KR08/00661

§ 371 Date: Aug. 13, 2009

(30) Foreign Application Priority Data
Feb. 15, 2007 (KR) 10-2007-0015709
May 14, 2007 (KR) 10-2007-0046516

(51) Int. Cl. A61K 47/10; A61K 47/02; A61K 47/12; A61K 47/36

(52) U.S. Cl. 514/770; 514/777; 514/782; 514/772; 514/784

(57) ABSTRACT

The present invention relates to a thixotropic pharmaceutical composition in which the viscosity is changed due to external mechanical stress applied so that isothermal and continuous gel/sol/gel transition occurs. The thixotropic pharmaceutical composition includes a pharmacologically active substance, a biocompatible thickener having a predetermined thixotropic property, and optionally a hydrophilic thickener. The viscosity of the composition is relatively rapidly changed within a predetermined range. Accordingly, it is easy to measure the amount of drugs to be administered, it is possible to administer a precise amount of drugs to a patient, the compliance of a patient with dosage of drugs is high, and it is easy to produce the composition.

### TECHNICAL FIELD

The present invention relates to a thixotropic pharmaceutical composition in which the viscosity changes according to external mechanical stress so that isothermal and continuous gel/sol/gel transition can occur. More particularly, the present invention relates to a thixotropic pharmaceutical composition that includes a pharmacologically active substance, a biocompatible thickener having a predetermined thixotropic property, and optionally a hydrophilic thickener. The viscosity of the composition is relatively rapidly changed within an appropriate range. Accordingly, it is easy to measure the amount of drugs to be administered, it is possible to administer a precise amount of drugs to a patient, the compliance of a patient with dosage of drugs is high, and it is easy to prepare the composition.

### BACKGROUND ART

Examples of pharmaceutical preparations for oral administration, which is used for systemic treatment, include solid agents including tablets and capsules, and liquid agents including syrups, elixirs, colloidal suspensions and the like. In the case of the solid agents, it is difficult to administer them to children, old persons, and persons that have a difficulty in swallowing. Accordingly, in order to solve the problem, oral liquid agents have been developed to increase the compliance of a patient with dosage of drugs and an absorption speed of drugs on the body.

However, the oral liquid agents have the following pharmaceutical and pharmacological problems. That is, in the case of the colloidal suspensions, during the storage, problems such as layer separation (caking and sedimentation) may occur in views of stability of the preparation. In the case of the syrups, due to the low viscosity and mechanical stress that is applied to a measuring tool (spoon or the like), there is a risk in that the drugs may be fallen from the measuring tool during the measuring and the oral administration. For example, in the case of the patient having dysmotility (the vibration of hand caused by the extremity tremor, the tremor of the hand, and the minute motion control shortage) or the pediatric patient having a fear about the taking medicine, it is very difficult to measure or administer the drugs in a precise amount by using the measuring tool.

In order to solve the above problems, many studies have been made to produce the semi-solid type preparation having the high viscosity by using a gel. For example, U.S. Pat. No. 5,300,302 discloses a gel type of preparation using a xanthan gum, a cellulose derivative and the like as a thickener for increasing the viscosity of the composition and a measuring container that can perform precise measuring pumping. U.S. Pat. Nos. 5,881,926, 6,071,523, 6,355,258, 6,399,079, and 6,656,482 disclose spill-resistant preparations (Trade name: ElixSure™, manufactured by Taro, Inc.). However, the preparations have the high viscosity and thus it is undesirably required to use high energy to disperse main drugs uniformly in a substrate during the manufacturing process. Furthermore, due to the limit on the viscosity range being capable of transition, there are problems that large external mechanical stress or a special container capable of measuring a precise amount is required during discharging a dosage from a container by pressing the container.

In addition, EP Pat. No. 0 379 147 discloses a gel that is capable of being discharged from a container provided with a unit measuring pump. However, there is a problem in that the gel can be taken in one day dosage only by pumping the content 12 to 60 times and repeating this procedure 3 or 4 times. The problem cannot be avoided simply by increasing the concentration of the pharmacologically active substance. The reason is that if the amount of the active substance is increased, the viscosity of the preparation is increased; accordingly, it is difficult to form the gel and to uniformly disperse the active substance in the substrate.

Moreover, the composition of the above-mentioned known technology is problematic in that due to the high viscosity characteristic, the manufacturing process is complicated and high energy is required.

Meanwhile, in U.S. Pat. Nos. 4,427,681 and 88/00825, a thixotropic substance is used as a suspension agent to avoid a structural characteristic of a cimetidine medicine, that is, the instability of the polymorph B type.

### DISCLOSURE OF INVENTION

### Technical Problem

The present invention has been made keeping in mind the problems occurring due to the high viscosity property of a semi-solid type preparation in the related art, and it is an object of the present invention to provide a thixotropic pharmaceutical composition that is easily produced, measured, and administered.

It is another object of the present invention to provide an oral pharmaceutical composition that has uniformly distributed pharmacologically active substances.

Hereinafter, the configuration and the operation of the present invention will be described in detail.

### Technical Solution

In order to accomplish the above objects, one aspect of the present invention is to provide a thixotropic pharmaceutical composition that comprises at least one pharmacologically active substance; a liquid substrate; and at least one biocompatible thickener having thixotropic property, and is characterized in that isothermal and continuous gel/sol/gel transition occurs when external mechanical stress is applied to the composition.

### BRIEF DESCRIPTION OF THE DRAWINGS

FIG. 1 is a graph that illustrates the hysteresis measurement results of a thixotropic pharmaceutical composition that is produced in Example 1;

FIG. 2 is a graph that illustrates the hysteresis measurement results of a thixotropic pharmaceutical composition that is produced in Example 3; and

FIG. 3 is a graph that illustrates the hysteresis measurement results of a thixotropic pharmaceutical composition that is produced in Example 4.

### BEST MODE FOR CARRYING OUT THE INVENTION

The initial viscosity that is defined in the present invention means viscosity when mechanical stress is not applied to a composition and the equilibrium viscosity that is defined in the present invention means viscosity in a state of in which mechanical stress is applied to a composition having the initial viscosity.

It is preferable that a pharmaceutical composition according to the present invention has the equilibrium viscosity of 4,000 cps or less when mechanical stress is applied such an extent that a container is being shaken. When the equilibrium viscosity is 4,000 cps or less as described above, it is easy to transport the pharmaceutical composition from a storage container to a measuring device, and is easy to perform the stirring, the filtration, or the packaging during the preparation process, thus the productivity can be improved.

It is more preferable that the composition has the initial viscosity of 5,000 cps or more in a static state and the equilibrium viscosity of 3,500 cps or less when the mechanical stress is applied thereto. It is even more preferable that the initial viscosity is in the range of 7,500 to 50,000 cps and the equilibrium viscosity is in the range of 300 to 3,500 cps.

It is most preferable that a difference between the initial viscosity and the equilibrium viscosity of the composition is at least 3,000 cps or more. More specifically, the difference may be in the range of 3,000 to 30,000 cps.

In connection with this, the initial viscosity and the equilibrium viscosity may be measured by using Rheometer RS 100 manufactured by Haake, Co., Ltd. at 25° C. and a shear rate of 30 rpm according to a PP35 plate/plate method.

The time taken up by transition from the equilibrium viscosity to the initial viscosity is in the range of 10 to 60 sec and preferably 10 to 30 sec.

In addition, when the composition according to the present invention is subjected to a spoon-overturning test in which a spoon overturns, the composition does not fall for 30 sec or more and preferably for 60 to 120 sec.

The composition according to the present invention can be measured and administered in a precise amount without spilling since a continuous gel/sol/gel transition phenomenon occurs. That is, the composition is present in a state of gel having the high and constant viscosity before the measuring (during the storage) and if the composition in a container is shaken to perform the measuring, the gel state is rapidly transitioned to the sol state having the low viscosity, accordingly, inconvenience of the measuring due to the high viscosity may be reduced. After the measuring, the sol state is reversibly transitioned to the gel state having the excellent internal cohesiveness, spread ability and the high viscosity in the measuring device to reduce the danger of the drug leakage from the measuring device, so that a precise amount of drugs can be administered. After the oral administration, it is easy to ensure the swallowing due to lowering viscosity caused by the body temperature and the saliva.

In addition, in the case of the composition according to the present invention, the initial viscosity is high, but the equilibrium viscosity is reduced due to external mechanical stress during the manufacturing. Thus, it is easy to produce, store, and pack the composition, and it is unnecessary to use high energy. Further, the reduced equilibrium viscosity is quickly increased again to the initial viscosity, accordingly, it is easy to measure a precise amount of composition and there is no danger of the drug leakage during the administration.

In the composition according to the present invention, the biocompatible thickener may be contained in an amount ranging from 0.01 to 12% (w/v) and preferably 0.01 to 5% (w/v) based on the total amount of the composition.

When the biocompatible thickener is contained in the above-mentioned amount, the composition of the present invention has an appropriate range of the viscosity so that it is possible to prevent the drug leakage from the measuring device, and as a result, it is easy to perform the preparation, the measuring, and the administration.

Examples of the biocompatible thickener may include any biocompatible thickener as long as the thickener has the thixotropic property, is biocompatible, and can be used as the thickener. Preferably, one selected from agar, carrageenan, carboxymethyl cellulose, a mixture of microcrystalline cellulose and carboxymethyl cellulose, colloidal silicon dioxide, xanthan gum, gellan gum, and a mixture thereof may be used.

In addition, the composition according to the present invention may further include one or more hydrophilic thickeners having no thixotropic property as the thickener. The hydrophilic thickener provides the viscosity that is enough to compensate the low viscosity of the biocompatible thickener having the thixotropic property to improve the viscosity and the thixotropic property of the composition according to the present invention. Thus, the viscosity, the thixotropic property and the like of the pharmaceutical composition according to the present invention may be optimized to be useful to perform the administration and the measuring.

The hydrophilic thickener may be contained in an amount ranging from 0.01 to 7% (w/v), preferably 0.01 to 3% (w/v), and more preferably 0.2 to 1% (w/v) based on the total amount of the composition.

Any thickener may be used as the hydrophilic thickener as long as the thickener has the hydrophilic property. Preferable examples of the thickener may include one selected from cellulose, hydroxypropylmethyl cellulose, hydroxyethyl cellulose, methyl cellulose, ethyl cellulose, hydroxypropyl cellulose, carboxymethyl cellulose, ethylhydroxyethyl cellulose, hydroxyethylmethyl cellulose, carboxymethylhydroxyethyl cellulose, alkylene oxide homopolymers including polyethylene oxide and polypropylene oxide, polyethylene glycol, alginates, polyvinyl alcohol, povidone, polysaccharides, vinyl pyrrolidone polymers, carboxyvinyl polymers, carboxy polymethylene, and a mixture thereof.

More preferable examples of the hydrophilic thickener include one selected from cellulose, carboxymethyl cellulose, alginate, polyethylene glycol, polyethylene oxide, polyvinyl alcohol, vinyl pyrrolidone polymers, carboxyvinyl polymers, carboxy polymethylene, povidone, and a mixture thereof.

In the composition according to the present invention, the type of pharmacologically active substance is not limited as long as the pharmacologically active substance can be orally administered to cure diseases, and one or more pharmacologically active substances that are known to be useful according to the type of disease in the art may be appropriately selected and determined by those who skilled in the art.

Examples of the above pharmacologically active substance may include one or more selected from analgesics, non-steroidal anti-inflammatory drugs, anti-histaminic agents, cough suppressants, expectorants, bronchodilators, anti-infective agents, CNS active drugs, cardiovascular drugs, anti-neoplastic drugs, cholesterol-lowering drugs, anti-emetics, vitamins, mineral supplementations, fecal softners, high blood pressure curing agents, anti-fungal agents, anti-diabetes drugs, amino acid agents and the like, but are not limited thereto.

More specific examples of the above pharmacologically active substance may include one or more selected from acetaminophen, ibuprofen, aspirin, diphenhydramine, valsartan, montelukast, scopolamine, zaltoprofen, tramadol, diclofenac, aceclofenac, etodolac, piroxicam, nimesulide, celecoxib, glucosamine, zolmitriptan, alendronic acid, risedreonic acid, S-carboxymethylcysteine, dextromethorphan, guaifenesin, pseudoephedrine, phenylephrine, loratadine, ephedrine, cetirizine, mizolastine, olopatadine, epinastine, procaterol, acetylcystein, erdostein, theophylline, formoterol, zipeprol, difemerine, tizanidine, tiropramide, diazepam, alprazolam, buspirone, etizolam, risperidone, olanzapine, amisulfiride, clomipramine, chlorpromazine, rivastigmine, donepezil, galantamine, entacapone, memantine, ropinirole, selegiline, carbidopa, levodopa, terfenadine, ranitidine, ciprofloxacin, triazolam, terbinafine, fluconazole, itraconazole, ketoconazole, voriconazole, propranolol, lovastatin, simvastatin, atorvastatin, pitavastatin, rosuvastatin, pravastatin, fenofibrate, ezetimibe, fluoxetine, enalapril, irbesartan, losartan, Ramipril, nicorandil, doxazosin, carvedilol, diltiazem, tropisetron, ondansetron, meclizine, azasetron, dolasetron, granisetron, metformin, glimepiride, gliclazide, mitiglinide, glibenclamide, repaglinide, and a pharmaceutically acceptable salt or ester thereof.

The content of the pharmacologically active substance is not limited, and may be appropriately determined in consideration of the type of disease, liquid substrate, and thickener. It is preferable that the pharmacologically active substance be contained in an amount ranging from 0.01 to 20% (w/v) based on the total amount of the composition.

In consideration of the type of pharmacologically active substance and thickener that are to be used, a solvent that is capable of appropriately dissolving or suspending the above substances may be used as the liquid substrate. Specific examples of the liquid substrate may be selected from purified water, glycerin, alcohols such as ethanol and the like, propylene glycol, polyethylene glycol, liquid polyols such as sorbitol, maltitol and the like, and a mixture thereof, but are not limited thereto. The liquid substrate may be contained in an amount ranging from 40 to 95% (w/v) based on the total amount of the composition.

Meanwhile, the composition according to the present invention may further include a sweetener and/or a flavor in order to prevent the bitter taste of the pharmacologically active substance. If the sweetener and the flavor having the fruit flavor that children like are added in a predetermined amount as described above, the bitter taste of the pharmacologically active substance may be completely prevented to improve the compliance of a pediatric patient with dosage of drugs. In addition, when a predetermined sweetener is used, the composition according to the present invention is administered to prevent the dental caries and does not affect the glucose content in the blood. Accordingly, the composition according to the present invention can be administered to diabetes patients without fear.

Examples of the sweetener may include a typical sweetener that is used in the art. Preferable examples of the sweetener may include one selected from sugar alcohols including mannitol, maltitol, sorbitol, xylitol, and isomalt that do not cause tooth decay, aspartames, acesulfames, saccharins, calcium saccharin, sodium saccharin, sucraloses, and a mixture thereof.

Examples of the flavor may include a typical flavor that is used in the art. Preferable examples of the flavor may include one or more selected from the group including flavors that have a chocolate flavor, a strawberry flavor, an orange essence, a grape flavor, a vanilla flavor, a cherry flavor, and an apple essence.

The sweetener and the flavor may be contained in an amount of 0.001 to 2% (w/v) based on the total amount of the composition, but the amount of the sweetener and the flavor is not limited thereto.

Furthermore, in the present invention, in order to prevent the bitter taste of the used pharmacologically active substance, a taste masking technology that is typically used in the art may be applied to the above pharmacologically active substance. For example, the pharmacologically active substance may be coated with a bio-degradable polymer, may be produced to have a solid dispersion substance form, or may be capsulated, but is not limited thereto. In connection with this, the coating of the biodegradable polymer, the production of the solid dispersion substance, and the encapsulation may be performed by using a method that is known in the art.

In addition, the composition according to the present invention may further include organic acids in order to improve the compliance of a patient with dosage of drugs. If the organic acids are added, the secretion of the saliva of the patient is promoted after the oral administration to reduce the viscosity of the composition. Thus, the convenience can be provided during the administration.

Examples of the organic acids that can be used in the present invention may include one or more selected from the group including a citric acid, an ascorbic acid, a palmitic acid, a tartaric acid and the like, but are not limited thereto.

The organic acids may be contained in an amount ranging from 0.001 to 5% (w/v) based on the total amount of the composition.

Additionally, according to the purpose and the case, excipients such as an preservative, a suspending agent, a solubilizing agent, a buffer agent and the like may be selected by those who skilled in the art and added thereto.

The thixotropic pharmaceutical composition according to the present invention may be produced by using a method of producing syrup or a semi-solid type preparation, which is well known in the art. Illustrative but non-limiting examples thereof will be described below: After a biocompatible thickener having the thixotropic property or a hydrophilic thickener is sufficiently hydrated by using an agitator, the pharmacologically active substance is separately dissolved or suspended to be mixed with the bio-compatible thickener or the hydrophilic thickener. Additionally, the liquid substrate, and the excipient such as a preservative, a buffer agent, a sweetener, an flavor, a colorant and the like are added thereto to be homogeneously mixed with each other, thereby performing the production. In connection with this, the excipient may be added thereto after being separately dissolved if necessary.

Furthermore, in the present invention, the thixotropic pharmaceutical composition may be stored in a storage container that can discharge the composition to a measuring device such as spoons. In connection with this, any container may be used as the above storage container as long as the container can be typically used to store or pack the pharmaceutical composition. Examples of the container may include a squeezable tube, a pumpable bottle, a pumpable squeezable tube, an individual pouch package and the like.

### MODE FOR THE INVENTION

The present invention will be described in more detail by the following Examples. However, the following Examples are illustrative only, and do not limit the scope of the present invention.

### Example 1

### Production of the Ibuprofen Oral Thixotropic Pharmaceutical Composition Using Carrageenan and Avicel CL 611 (100 ml)

45 g of xylitol, 10 g of the D-sorbitol solution, and 10 g of glycerin were added to 40 g of the purified water, stirred and mixed with each other. 1.5 g of carrageenan and 0.500 g of Avicel CL 611 were added thereto while the resulting solution was continuously stirred to perform the hydration, and 0.25 g of the citric acid and 0.1 g of the sodium benzoate were added to perform the solubilization (A). After 0.1 g of Twin 80 was separately added to 10 g of the purified water to be dissolved therein, 2 g of ibuprofen was added thereto, and the stir and the suspension were performed (B). After A was added to B, stirred, and mixed with each other, 0.075 g of Yellow No. 5 and 0.1 g of orange essence were sequentially added thereto and mixed with each other, and the purified water was added thereto so that the total volume of the resulting solution was 100 ml.

TABLE 1

| Part | Ingredient | Quantity (g) |
|---|---|---|
| A | Purified water | 40.000 |
| | Xylitol | 45.000 |
| | D-sorbitol solution | 10.000 |
| | glycerin | 10.000 |
| | Carrageenan | 1.500 |
| | Avicel CL 611 | 0.500 |
| | Citric acid | 0.250 |
| | Sodium benzoate | 0.100 |
| B | Purified water | 10.000 |
| | Twin 80 | 0.100 |
| | Ibuprofen | 2.000 |
| | Yellow No. 5 | 0.075 |
| | Orange essence | 0.100 |
| | Purified water | Predetermined amount |

### Example 2

### Production of the S-carboxymethyl Cystein Oral Thixotropic Composition Using Aerosil 200 and HPMC 2906 (100 ml)

25 g of white sugar and 10 g of the D-sorbitol solution were added to 50 g of the purified water, stirred and mixed with each other. 1.5 g of HPMC 2906 and 2 g of Aerosil 200 were added to the resulting solution, stirred, and hydrated (A). After 0.37 g of sodium hydroxide was separately added to 15 g of the purified water to be dissolved therein, 2 g of S-carboxymethyl cystein was added thereto and then dissolved therein (B). A was added to B, stirred, and mixed with each other. 0.09 g of methyl paraben and 0.01 g of propyl paraben were separately added to 1.5 g of ethanol and then dissolved therein (C). After C was added to the solution mixture of A and B and mixed, 0.1 g of Red No. 40 and 0.1 g of the essence having the strawberry flavor were added thereto and the mixing was uniformly performed, and the purified water was added thereto so that the total volume of the resulting solution was 100 ml.

TABLE 2

| Part | Ingredient | Quantity (g) |
|---|---|---|
| A | Purified water | 50.000 |
| | White sugar | 25.000 |
| | D-sorbitol solution | 10.000 |
| | HPMC 2906 (6000 cp) | 1.500 |
| | Aerosil 200 | 2.000 |
| B | Purified water | 15.000 |
| | Sodium hydroxide | 0.370 |
| | S carboxymethyl cystein | 2.000 |
| C | Ethanol | 1.500 |
| | Methyl paraben | 0.090 |
| | Propyl paraben | 0.010 |
| | Red No. 40 | 0.100 |
| | Essence having the | 0.100 |
| | strawberry flavor | |
| | Purified water | Predetermined amount |

### Example 3

### Production of the Ibuprofen Oral Thixotropic Composition Using Carrageenan (100 ml)

45 g of xylitol, 10 g of the D-sorbitol solution, and 10 g of glycerin were added to 40 g of the purified water, stirred and mixed with each other. 1.5 g of carrageenan was added thereto while the resulting solution was continuously stirred to perform the hydration, and 0.25 g of the citric acid and 0.1 g of the sodium benzoate were added to perform the solubilization (A). After 0.1 g of Twin 80 was separately added to 10 g of the purified water to be dissolved therein, 2 g of ibuprofen was added thereto, and the stir and the suspension were performed (B). After A was added to B, stirred, and mixed with each other, 0.075 g of Yellow No. 5 and 0.1 g of orange essence were sequentially added thereto and mixed with each other, and the purified water was added thereto so that the total volume of the resulting solution was 100 ml.

TABLE 3

| Part | Ingredient | Quantity (g) |
|---|---|---|
| A | Purified water | 40.000 |
| | Xylitol | 45.000 |
| | D-sorbitol solution | 10.000 |
| | glycerin | 10.000 |
| | Carrageenan | 1.500 |
| | Citric acid | 0.250 |
| | Sodium benzoate | 0.100 |
| B | Purified water | 10.000 |
| | Twin 80 | 0.100 |
| | Ibuprofen | 2.000 |
| | Yellow No. 5 | 0.075 |
| | Orange essence | 0.100 |
| | Purified water | Predetermined amount |

### Example 4

### Production of the Ibuprofen Oral Thixotropic Composition Using Gellan Gum and Carrageenan (100 ml)

After 0.08 g of methyl paraben and 0.02 g of butyl paraben were dissolved in 40 g of the hot purified water (about 80° C.), the resulting solution was cooled at the room temperature. 15 g of the D-sorbitol solution and 15 g of glycerin were added thereto, stirred and mixed with each other. 0.26 g of carrageenan and 1.0 g of gellan gum were added thereto while the resulting solution was continuously stirred to perform the hydration, and 0.144 g of the citric acid anhydrous and 0.137 g of sodium citrate dihydrate were added to perform the solubilization (A). After 0.1 g of Poloxamer was separately added to 10 g of the purified water to be dissolved therein, 2 g of ibuprofen was added thereto, and the stir and the suspension were performed (B). After A was added to B, stirred, and mixed with each other, 0.14 g of sucralose, 0.01 g of Red No. 40 and 0.3 g of the strawberry flavor were sequentially added thereto and mixed with each other, and the purified water was added thereto so that the total volume of the resulting solution was 100 ml.

TABLE 4

| Part | Ingredient | Quantity (g) |
|---|---|---|
| A | Purified water | 40.000 |
| | D-sorbitol solution | 15.000 |
| | glycerin | 15.000 |
| | Carrageenan | 0.260 |
| | Gellan gum | 1.000 |
| | Citric acid anhydrous | 0.144 |
| | Sodium citrate dihydrate | 0.137 |
| | Methyl paraben | 0.080 |
| | Butyl paraben | 0.020 |
| B | Purified water | 10.000 |
| | Poloxamer | 0.100 |
| | Ibuprofen | 2.000 |
| | Sucralose | 0.140 |
| | Red No. 40 | 0.010 |
| | Strawberry flavor | 0.300 |
| | Purified water | Predetermined amount |

### Example 5

### Production of the Ibuprofen Oral Thixotropic Composition Using Xanthan Gum, Gellan Gum and Carrageenan (100 ml)

After 0.08 g of methyl paraben and 0.02 g of butyl paraben were dissolved in 40 g of the hot purified water (about 80° C.), the resulting solution was cooled at the room temperature. 10 g of the D-sorbitol solution and 10 g of glycerin were added thereto, stirred and mixed with each other. 0.26 g of carrageenan, 0.6 g of gellan gum, and 0.15 g of xanthan gum were added thereto while the resulting solution was continuously stirred to perform the hydration, and 0.144 g of the citric acid anhydrous and 0.137 g of sodium citrate dihydrate were added to perform the solubilization (A). After 0.1 g of Poloxamer was separately added to 10 g of the purified water to be dissolved therein, 2 g of ibuprofen was added thereto, and the stir and the suspension were performed (B). After A was added to B, stirred, and mixed with each other, 0.14 g of sucralose, 0.01 g of Red No. 40 and 0.3 g of the strawberry flavor were sequentially added thereto and mixed with each other, and the purified water was added thereto so that the total volume of the resulting solution was 100 ml.

TABLE 5

| Part | Ingredient | Quantity (g) |
|---|---|---|
| A | Purified water | 40.000 |
| | D-sorbitol solution | 10.000 |
| | glycerin | 10.000 |
| | Carrageenan | 0.260 |
| | Gellan gum | 0.600 |
| | Xanthan gum | 0.150 |
| | Citric acid anhydrous | 0.144 |
| | Sodium citrate dihydrate | 0.137 |
| | Methyl paraben | 0.080 |
| | Butyl paraben | 0.020 |
| B | Purified water | 10.000 |
| | Poloxamer | 0.100 |
| | Ibuprofen | 2.000 |
| | Sucralose | 0.140 |
| | Red No. 40 | 0.010 |
| | Strawberry flavor | 0.300 |
| | Purified water | Predetermined amount |

### Experimental Example 1

### First Evaluation of External Mechanical Stress-Resistance (Spoon Vibration Evaluation; Vibration Test)

The resistances of the preparations of Examples 1 to 5 and Elixsure™ that was the commercial semi-solid dosage form in respect to external mechanical stress were evaluated. This was performed by using Vortex Genie2 manufactured by Scientific Industries, Co. 2 g of each preparation was put on the spoon having the long diameter of 35 mm and the short diameter of 25 mm of the spatula having the length of 150 mm, and the opposite side of the spoon was fixed to the rotation plate of the device. The spoon was rotated and vibrated in a weak intensity (Vortex 3) and in a strong intensity (Vortex 10), and the time when the preparation was discharged from the spoon was measured to perform the evaluation. The results are described in the following Table 6.

TABLE 6

| | Vortex 3 (sec) | Vortex 10 (sec) |
|---|---|---|
| Example 1 | >120 | >60 |
| Example 2 | >120 | >60 |
| Example 3 | >100 | >50 |
| Example 4 | >110 | >55 |
| Example 5 | >120 | >60 |
| ElixSure | >60 | >40 |

### Experiment Example 2

### Second Evaluation of External Mechanical Stress-Resistance (Spoon Overturning Test)

In order to perform another evaluation of the resistances of the preparations of Examples 1 to 5 and ElixSure™ that was the commercial preparation in respect to external mechanical stress, the spoon overturning test was performed. After the each preparation was put on the spoon that had the length of 90 mm, the long diameter of 35 mm and the short diameter of 28 mm and the surface of the preparation was made flat, the spoon was overturned and the time when the preparation was fallen from the spoon was measured to perform the evaluation. The results are described in the following Table 7.

TABLE 7

| | Overturning Time (sec) |
|---|---|
| Example 1 | >120 |
| Example 2 | >120 |
| Example 3 | >110 |
| Example 4 | >120 |
| Example 5 | >120 |
| ElixSure | >30 |

From Experimental Examples 1 and 2, it could be seen that the composition according to the present invention had the high initial viscosity and thus the measuring and the administration were easily performed.

### Experiment Example 3

### First Evaluation of the Thixotropic Property (Measurement of a Change in Viscosity in Respect to External Mechanical Stress)

The thixotropic properties of the preparations of Examples 1 to 5 and ElixSure™ that was the commercial preparation were evaluated. This was performed by using Rheometer RS 100 manufactured by Hakke, Co., Ltd (25° C., PP35 plate/plate method, and shear rate of 30 rpm). The viscosity of the sample that was left for 1 day later the production (viscosity 1, initial viscosity) was measured. Also, after the same sample was put into 100 ml of the graduated cylinder and the tapping was repeated 100 times by using the tapping machine, the viscosity of the sample (viscosity 2, equilibrium viscosity) was measured. The results are described in the following Table 8.

TABLE 8

| | Viscosity 1 | Viscosity 2 |
|---|---|---|
| | (initial | (equilibrium |
| | viscosity, cps) | viscosity, cps) |
| Example 1 | >7,000 | <4,000 |
| Example 2 | >7,500 | <4,000 |
| Example 3 | >7,000 | <4,000 |
| Example 4 | >8,000 | <4,000 |
| Example 5 | >8,500 | <4,000 |
| ElixSure | >6,000 | >5,000 |

From Experimental Example 3, it could be seen that in the case of the composition according to the present invention, a difference between the initial viscosity and the equilibrium viscosity was 3000 cps or more.

### Experimental Example 4

### Second Evaluation of the Thixotropic Property (Measurement of a Change in Flow Ability in Respect to External Mechanical Stress)

The thixotropic properties of the preparations of Examples 1 to 5 and ElixSure™ that was the commercial preparation were evaluated. This was performed by measuring the time required that a predetermined amount (100 ml) of sample was passed through the funnel having the tube having the diameter of 2 cm. The passing time of the sample that was left for 1 day later the production (time 1) was measured. Also, after the same amount of the same sample was put into 200 ml of the graduated cylinder and the tapping was repeated 100 times by using the tapping machine, the passing time of the sample (time 2) was measured. The results are described in the following Table 9.

TABLE 9

| | Time 1 (sec) | Time 2 (sec) |
|---|---|---|
| Example 1 | >60 | <10 |
| Example 2 | >60 | <10 |
| Example 3 | >50 | <10 |
| Example 4 | >55 | <10 |
| Example 5 | >60 | <15 |
| ElixSure | >60 | >50 |

From the above Experimental Example 4, it could be seen that when external mechanical stress was applied to the composition according to the present invention, the viscosity of the composition was reduced, thus reducing the funnel passing time.

### Experiment Example 5

### Third Evaluation of the Thixotropic Property (Measurement of the Viscosity Recovering Speed)

The thixotropic properties of the preparations of Examples 1 to 5 and ElixSure™ that was the commercial preparation were evaluated. The increase in viscosity that had been reduced due to a predetermined external mechanical stress according to the time was evaluated by using a spoon overturning test (see Experimental Example 2). The sample that was left for 1 day after the production was put into 200 ml of the graduated cylinder and the overturning time was measured immediately (time 1) after the tapping was repeated 100 times by using the tapping machine, at 10 sec (time 2) after the tapping was repeated 100 times by using the tapping machine, at 30 sec (time 3) after the tapping was repeated 100 times by using the tapping machine, and at 60 sec (time 4) after the tapping was repeated 100 times by using the tapping machine. The results are described in the following Table 10.

TABLE 10

| | Time 1 (sec) | Time 2 (sec) | Time 3 (sec) | Time 4 (sec) |
|---|---|---|---|---|
| Example 1 | >10 | >60 | >120 | >120 |
| Example 2 | >10 | >60 | >120 | >120 |
| Example 3 | >10 | >50 | >100 | >110 |
| Example 4 | >10 | >60 | >110 | >120 |
| Example 5 | >20 | >70 | >120 | >120 |
| ElixSure | >25 | >25 | >25 | >30 |

From the above Experimental Example 5, it could be seen that the viscosity of the composition according to the present invention began to be increased from 10 sec after removing the external mechanical stress and was recovered to be similar to the initial viscosity at 30 sec after the mechanical stress was removed from system. Thus, the results of the spoon overturning test were similar to the results of the initial viscosity.

### Experiment Example 6

### Fourth Evaluation of the Thixotropic Property (Measurement of Hysteresis)

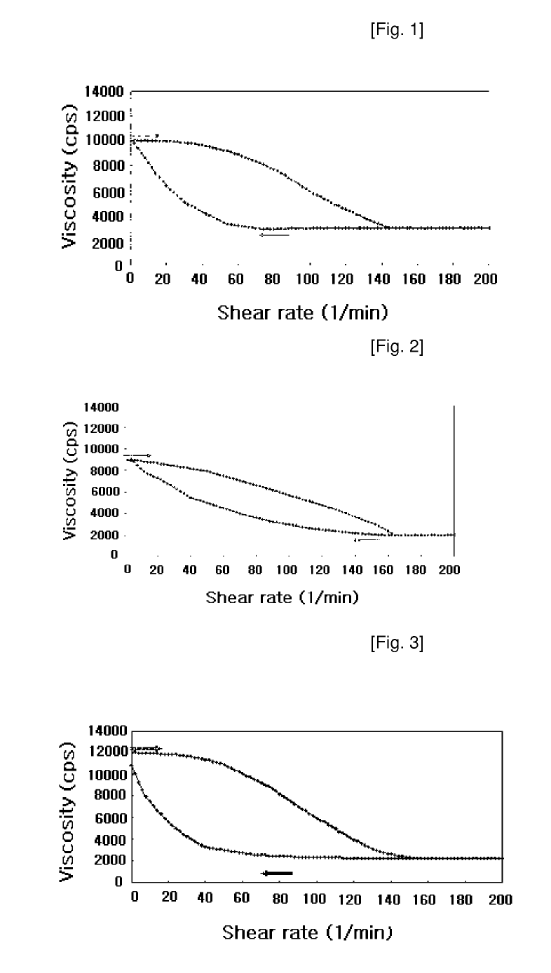

The thixotropic properties of the preparations of Examples 1, 3, and 4 and ElixSure™ were evaluated. The hysteresis of each of the samples was obtained by using Rheometer RS 100 manufactured by Haake, Co., Ltd. (25° C., PP35 plate/plate, and hysteresis analysis method). While the shear rate was increased from 0 rpm to 200 rpm for 60 min and then reduced from 200 rpm to 0 rpm at the same rate, the viscosity was measured and the results were expressed by using the graphs. The results are shown in FIGS. 1 to 3.

### INDUSTRIAL APPLICABILITY

As described above, the thixotropic pharmaceutical composition according to the present invention includes one or more types of biocompatible thickeners having the thixotropic property and optionally a hydrophilic thickener that does not have the thixotropic property. Since the viscosity of the composition is continuously and rapidly changed in a predetermined range, it is possible to administer a precise amount of drugs, the compliance of a patient with dosage of drugs is high, and it is easy to produce the composition.

1. A thixotropic pharmaceutical composition that is a drug delivery composition comprising:
at least one pharmacologically active substance;
a liquid substrate; and
at least one biocompatible thickener having a thixotropic property,
and having an equilibrium viscosity of 4,000 cps or less.

2. The thixotropic pharmaceutical composition according to claim 1, wherein a difference between an initial viscosity and the equilibrium viscosity is at least 3,000 cps or more.

3. The thixotropic pharmaceutical composition according to claim 1, wherein a time taken up by transition from the equilibrium viscosity to the initial viscosity is in the range of 10 sec to 30 sec.

4. The thixotropic pharmaceutical composition according to claim 1, wherein the biocompatible thickener is included in an amount ranging from 0.01% (w/v) to 12% (w/v).

5. The thixotropic pharmaceutical composition according to claim 4, wherein the biocompatible thickener is included in an amount ranging from 0.01% (w/v) to 5% (w/v).

6. The thixotropic pharmaceutical composition according to claim 1, wherein the biocompatible thickener is one or more selected from the group consisting of an agar, a carrageenan, a gellan gum, colloidal silicon dioxide, and a xanthan gum.

7. The thixotropic pharmaceutical composition according to claim 1, further comprising one or more selected from the group consisting of a sweetener and a flavor.

8. The thixotropic pharmaceutical composition according to claim 7, wherein the sweetener is one or more selected from the group consisting of sugar alcohols including mannitol, maltitol, sorbitol, xylitol, and isomalt that do not cause a tooth decay, aspartames, acesulfames, saccharins, calcium saccharin, sodium saccharin, and sucraloses.

9. The thixotropic pharmaceutical composition according to claim 1, further comprising organic acids.

10. The thixotropic pharmaceutical composition according to claim 9, wherein the organic acids are one or more selected from the group consisting of a citric acid, an ascorbic acid, a palmitic acid, and a tartaric acid.

11. The thixotropic pharmaceutical composition according to claim 1, further comprising a hydrophilic thickener.
